(19) United States
(12) Patent Application Publication
(10) Pub. No.: US 2015/0126608 A1
(43) Pub. Date: May 7, 2015

(54) TREATMENT OF PREADOLESCENT MODERATE ACNE VULGARIS

(71) Applicant: GALDERMA RESEARCH & DEVELOPMENT, Biot (FR)

(72) Inventors: Lawrence F. Eichenfield, San Diego, CA (US); Zoe D. Draelos, High Point, NC (US); Anne W. Lucky, Cincinnati, OH (US); Adelaide A. Herbert, Houston, TX (US); Jeffrey Sugarman, Santa Rosa, CA (US); Linda Stein Gold, Bloomfield Hills, MI (US); Diane Rudisill, Princeton Junction, NJ (US); Hong Liu, Perrineville, NJ (US); Vasant Manna, Ewing, NJ (US)

(21) Appl. No.: 14/397,728

(22) PCT Filed: May 24, 2013
(86) PCT No.: PCT/EP2013/060805

§ 371 Date: Oct. 29, 2014

Related U.S. Application Data
(60) Provisional application No. 61/652,048, filed on May 25, 2012.

(51) Int. Cl. A61K 31/327; A61K 9/00; A61K 47/10; A61K 47/26; A61K 47/20; A61K 31/192; A61K 47/32

(52) U.S. Cl. CPC A61K 31/327; A61K 31/192; A61K 9/0014; A61K 47/32; A61K 47/26; A61K 47/20; A61K 47/10
USPC 514/569

(57) ABSTRACT

A method of treating preadolescent acne is described. The method can include administering an effective amount of a pharmaceutical composition that includes a fixed combination of adapalene and benzol peroxide to a preadolescent patient in need thereof.

The preadolescent patient can be from age 7 to age 11, and the pharmaceutical composition can include from 0.01% to 2% by weight of adapalene and from 0.1% to 5% by weight by weight of benzol peroxide.

Also described, is a composition for use in such a method.

### TECHNICAL FIELD

The treatment of preadolescent moderate acne vulgaris with a fixed combination of adapalene and of benzoyl peroxide (hereafter “BPO”) is described.

6-[3-(1-Adamantyl)-4-methoxyphenyl]-2-naphthoic acid (referred to hereinbelow as adapalene) is a naphthoic acid derivative with retinoid and anti-inflammatory properties. This molecule was developed for the topical treatment of common acne and of dermatoses sensitive to retinoids.

### BACKGROUND

Adapalene is marketed under the trademark Differin® at a weight concentration of 0.1%, in the form of an “alcoholic lotion” solution, an aqueous gel and a cream. These compositions are useful for treating acne.

FR 2,837,101 describes adapalene compositions at a weight concentration of 0.3%, for treating acne.

WO 03/0555 472 and corresponding US 2003/0170196 moreover describe stable pharmaceutical compositions comprising adapalene and benzoyl peroxide (BPO).

U.S. Pat. No. 8,080,537 describes the treatment of acne vulgaris with a fixed combination of adapalene and BPO in a group of patients having a minimum age of 12.5 and a mean age of 18.7. This adolescent and post-adolescent population experienced decreased inflammatory lesions to a greater extent than the decrease in non-inflammatory lesions.

As the onset of acne tends to be earlier, there is a need for pediatricians and young patients to have appropriate treatments for acne.

It has now been demonstrated, surprisingly, that in a population of pre-adolescent acne patients who typically have a preponderance of non-inflammatory lesions, a fixed combination of adapalene and BPO according to the invention, can provide an unexpectedly large decrease in the numbers of non-inflammatory acne lesions and an improvement in the clinical condition of pre-adolescent patients that is markedly superior to those of a treatment based on adapalene alone or on BPO alone, while at the same time maintaining the same skin tolerance.

The said treatment takes advantageously the form of a pharmaceutical composition combining fixed amounts of adapalene and BPO.

Hence in one embodiment of invention, it is provided a method of treating preadolescent acne, the method comprising administering an effective amount of a pharmaceutical composition comprising a fixed combination of adapalene and BPO to a preadolescent patient in need thereof, wherein the preadolescent patient is from age 7 to age 11, and wherein the pharmaceutical composition comprises from 0.01% to 2% by weight of adapalene and from 0.1 to 5% by weight by weight of benzol peroxide.

According to a particularly preferred embodiment of the invention, the pharmaceutical composition comprises 0.1% to 0.3% by weight adapalene and 2.5% by weight BPO.

In one particular embodiment, said pharmaceutical composition is formulated as a topical gel.

Accordingly, the method results in a treatment success are of at least 49%. Similarly, the method provides improved treatment success when compared with vehicle alone as early as week 4 of treatment.

Comparably, the method provides:
results in a reduction in total lesion count of at least 68%
results in a reduction of inflammatory lesion count of at least 63% and/or
results in a reduction of non-inflammatory lesion count of at least 70%.

According said method also, the total, inflammatory and non-inflammatory lesions results were significantly superior as early as week 1 when compared with vehicle alone.

Another embodiment of the invention provides a method of treating non-inflammatory acne lesions in a preadolescent patient, the method comprising topically administering to the skin of the preadolescent patient an effective amount of a fixed combination comprising from about 0.1% to about 0.3% by weight adapalene and about 2.5% by weight BPO, wherein the preadolescent patient is from age 7 to age 11 and wherein the amount of the fixed combination administered is effective to result in a reduction of non-inflammatory lesion count of at least about 70%.

The present invention also concerns a composition for use the method as disclosed herein. The present invention therefore concerns a pharmaceutical composition comprising a fixed combination of adapalene and benzol peroxide, for its use in a method of treating preadolescent acne, which comprises administering an effective amount of said pharmaceutical composition to a preadolescent patient in need thereof, wherein the preadolescent patient is from age 7 to age 11, and wherein the pharmaceutical composition comprises from 0.01% to 2% by weight of adapalene and from 0.1 to 5% by weight by weight of benzol peroxide.

The detailed description hereafter applies to the method as well as to the pharmaceutical composition of the present invention.

Exemplary embodiments feature formulation of adapalene or a pharmaceutically acceptable salt thereof into a pharmaceutical composition, especially at set doses, intended to be administered in combination with benzoyl peroxide (BPO), for the treatment of non-inflammatory and/or other acne lesions, especially to reduce the number of non-inflammatory acne lesions and to thus improve the clinical condition of pre-adolescent patients.

### DETAILED DESCRIPTION

Other than in the operating examples, or where otherwise indicated, all numbers expressing quantities of ingredients and/or percentages of improvement (such as reduction of number of lesions) are to be understood as being modified in all instances by the term “about,” meaning within ±10% of the indicated number.

Acne is initially characterized by keratinization disorders, which are sometimes invisible to the naked eye. Visible acne lesions then develop, while the size of the sebaceous glands and the production of sebum increase.

Exemplary embodiments specifically concern acne lesions. The term “acne lesions” means non-inflammatory lesions (open and closed comedones) and inflammatory lesions (papules, pustules, nodules and cysts) caused by acne. In pre-adolescent, non-inflammatory lesions predominate, since hormonal changes are not yet present to contribute to the development of inflammatory lesions.

More preferably, the fixed pharmaceutical combination is administered by daily cutaneous topical application.

The term “adapalene salts” means the salts of adapalene formed with a pharmaceutically acceptable base, especially mineral bases such as sodium hydroxide, potassium hydroxide and ammonia or organic bases such as lysine, arginine or N-methylglucamine. The term “adapalene salts” also means the salts formed with fatty amines such as dioctylamine and stearylamine.

The expression “combination of adapalene or salts thereof with benzoyl peroxide” means a single composition comprising both adapalene or salts thereof and benzoyl peroxide.

According to the invention, the pharmaceutical composition is a fixed combination and comprises, in a pharmaceutically acceptable medium, (i) at least one compound selected from among adapalene and pharmaceutically acceptable salts thereof, and (ii) benzoyl peroxide (BPO). Preferably, the pharmaceutical composition is intended for a single topical application per day.

The term “pharmaceutically acceptable medium” means a medium that is compatible with the skin, mucous membranes and the integuments.

The term “fixed combination” should be understood as meaning a combination whose active principles are combined at fixed doses in the same vehicle (single formula) that delivers them together to the point of application. Preferably, the pharmaceutical composition in the form of a fixed combination is a gel; in this case, the two active principles are dispersed and intimately mixed, during production, in the same vehicle, which delivers them together during the application of the gel.

The Lesions counts and the Success are rated using an Investigator's Global Assessment (IGA) scale. In general, a minimum treatment duration is needed to demonstrate efficacy in acne trials investigating topical products.

The Lesions counts consider the two major types of acne lesions: non-inflammatory and inflammatory. Non-inflammatory lesions of acne are the open (blackheads) or closed (whiteheads) comedones. Inflammatory lesions are divided into papules, pustules, and nodules/nodulocystic lesions, depending on the severity and location of the inflammation within the dermis

Based on an IGA, the Success rate typically represents the proportion of patients achieving “Clear” or “almost clear” as show in table below. Those patients would not need further treatment as shown in pictures following.

The treatments have a variable duration, depending on the patient and the severity of his/her acne. The treatment period may thus run from several weeks to several months. A suitable treatment period is at least two weeks, preferably from 2 weeks to 24 weeks. In a preferred embodiment duration treatment is from 4 to 12 weeks. The treatment duration however can be adapted by the physician who can decide to stop the treatment at week 2, week 4, week 5, week 6, week 7, week 8, week 9 or week 10 according to the excellent results obtained. In other words, treatment duration can last from 1 month to 6 months and more preferably a duration of 3 months is preferable, the duration of the treatment possibly being prolonged, if necessary.

According to one embodiment, the invention provides prompt treatment of acne with a fast onset of action. This property helps to not only prevent scarring, but also later severity progression in older childhood and adolescent years. This early onset of action is apparent as soon as the first week of treatment as shown on FIG. 3. Indeed, for total, inflammatory and non-inflammatory lesions, results were significantly superior for adapalene-BPO as early as week 1 compared to vehicule, and remained significant at all time points (FIG. 3).

Patients are often recruited for study entry at their worst severity and usually improve during the course of therapy, whether the therapy is active or placebo (vehicle in the case of topical products). The benefit of an active is therefore demonstrated if the improvement reported under treatment is greater to the one observed under placebo or vehicule.

All the pharmaceutical compositions administered in accordance with the invention can comprise from 0.01% to 2% by weight, preferably from 0.05% to 0.5% by weight and more preferentially from 0.1% to 0.3% by weight of adapalene.

The composition comprises also BPO, in an amount that can range from 0.1% to less than 5% by weight and preferably from 0.5% to less than 5% by weight of BPO, more preferably from 2% to less than 5% by weight of BPO and more preferentially 2.5% by weight of BPO.

All the percentages of amounts of ingredients in the present description are indicated by weight relative to the total weight of the composition.

In a particularly preferred embodiment, the pharmaceutical composition comprises from 0.1% to 0.3% by weight of adapalene and 2.5% by weight of BPO, and more preferably 0.1% of adapalene and 2.5% of BPO.

The pharmaceutical compositions according to the invention can be in the form of ointments, emulsions preferably in the form of creams, milks or pomades; powders, impregnated pads, solutions, gels, sprays, lotions or suspensions. They can also be in the form of suspensions of microspheres or nanospheres or of lipid or polymer vesicles or of polymer patches and/or of hydrogels allowing controlled release. These compositions can be in anhydrous form, in aqueous form or in the form of an emulsion.

In one preferred embodiment of the invention, the pharmaceutical compositions are in the form of a gel, a cream or a solution referred to as a lotion.

Preferably, the pharmaceutical compositions combining adapalene and BPO are gels.

The pharmaceutical compositions of the invention can contain inert additives or combinations of these additives, such as:
wetting agents;
texture enhancers;
preservatives such as para-hydroxybenzoic acid esters;
stabilizers;
humidity regulators;
pH regulators;
osmotic pressure modifiers;
emulsifiers;
UV-A and UV-B screening agents; and
antioxidants, such as a-tocopherol, butylhydroxyanisole or butylhydroxytoluene, superoxide dismutase, ubiquinol, or certain metal-chelating agents.

Needless to say, one skilled in this art will take care to select the optional compound(s) to be added to these compositions such that the advantageous properties intrinsically associated with the present invention are not, or are not substantially, adversely affected by the envisaged addition.

According to one particular embodiment, the pharmaceutical composition can be an aqueous gel especially containing one or more ingredients selected from among the carbomer 940 (BF Goodrich Carbopol 980), acrylamide and sodium acryloyldimethyltaurate copolymer and isohexadecane and polysorbate 80 (Simulgel 600) and propylene glycol, or a cream especially containing one or more ingredients selected from among perhydrosqualene, cyclomethicone, PEG-20 methylglucose sesquistearate and methylglucose sesquistearate or an “alcoholic lotion” solution based on polyethylene glycol.

Useful pharmaceutical compositions, comprising adapalene and BPO, are moreover described in WO 03/055 472. Examples of such compositions comprise, besides the active principles adapalene and BPO:
from 5% to 25% of water;
from 0% to 10%, preferably from 0% to 2% and preferably less than 0.5% of liquid wetting surfactant;
from 0% to 10% of pro-penetrating agent; and
an aqueous phase comprising a pH-independent gelling agent.
   According to one preferred embodiment, the preferred pharmaceutical composition, comprising adapalene and BPO, is an aqueous gel having the following formulation:
2.5% of BPO;
0.1% of adapalene;
0.10% of disodium EDTA;
4.00% of glycerol;
4.00% of propylene glycol;
   and also, preferably:
0.05% of sodium docusate;
0.20% of poloxamer 124;
4.00% of acrylamide and sodium acryloyldimethyltaurate copolymer and isohexadecane and polysorbate 80;
NaOH, in an amount sufficient to obtain a pH of 5.

### BRIEF DESCRIPTION OF THE DRAWINGS

FIG. 1 is a flow chart showing subject dispositions and demographics for the study described in the example hereafter;

FIG. 2 is a graph of success rate, in percent, for a pharmaceutical composition comprising a fixed combination of adapalene and BPO compared to vehicle, for the time course of the study;

FIG. 3 is a graph showing the medium percentage change, in percent, from baseline, in (A) total lesions, (B) inflammatory lesions and (C) non-inflammatory lesions, over the time course of the study.

FIG. 4 is a pair of photographs of an 11-year old male patient at (A) baseline and (B) after treatment for 12 weeks with a pharmaceutical composition comprising a fixed combination of adapalene and BPO;

FIG. 5 is a pair of photographs of an 11-year old female patient at (A) baseline and (B) after treatment with a pharmaceutical composition comprising a fixed combination of adapalene and BPO;

FIG. 6 is a graph of parent assessment of their child's acne after treatment with a pharmaceutical composition comprising a fixed combination of adapalene and BPO, compared to treatment with vehicle; and

FIG. 7 is a graph showing mean scores of all tolerability signs (erythema, scaling, dryness and stinging/burning) over the course of the study with a pharmaceutical composition comprising a fixed combination of adapalene and BPO, compared to vehicle.

### EXAMPLE

### Results of a Clinical Study

The following example presents results obtained in a clinical study.
The following abbreviations are used hereafter:
Adapalene-BPO: adapalene 0.1%-benzoyl peroxide 2.5%
AE: adverse event

### C-DLQI: Children's Dermatology Life Quality Index

### GCP: Good Clinical Practice

### ICH: International Conference on Harmonization

### IGA: Investigator's Global Assessment

P. acnes: Propionibacterium acnes
The topical gel used in the study had the following formulation (Epiduo® gel) in which percents are expressed by weight with regard to the total weight of the composition:

| | |
|---|---|
| Adapalene | 0.10% |
| Benzoyl peroxide | 2.50% |
| Copolymer of acrylamide and sodium | 4.00% |
| acryloyldimethyltaurate (in isohexadecane and | |
| polysorbate 80) | |
| Sodium docusate | 0.05% |
| Glycerol | 4.00% |
| Poloxamer (e.g., poloxamer 124) | 0.20% |
| Propylene glycol | 4.00% |
| Purified water | qs 100% |

An increasingly earlier onset of acne vulgaris is seen among preadolescents, yet most medications are indicated for patients at least 12 years old. Controlling acne early may reduce the risk of scarring and avoid worsened disease severity in the future.

Adapalene-benzoyl peroxide fixed-dose combination gel was effective and well-tolerated in treating acne among 9-11 year-olds, leading to significantly superior treatment success and reduced lesion counts compared to vehicle, good parent evaluation and trends toward an improved quality of life.

An evaluation of the efficacy and safety of adapalene 0.1%-benzoyl peroxide 2.5% gel (adapalene-BPO) in patients 9-11 years old with acne vulgaris is described herein.

Enrolled subjects were male or female, with a score of 3 (moderate) on the Investigator's Global Assessment (IGA) scale. Subjects were randomized to receive adapalene-BPO or vehicle once daily for up to 12 weeks. Efficacy was evaluated by success rate (percentage of subjects rated “clear” or “almost clear”) at each visit, median percentage changes from baseline in total, inflammatory and non-inflammatory lesion counts at each visit, the Children's Dermatology Life Quality Index (C-DLQI) at baseline and week 12, and the Parent Assessment of Acne at week 12. Safety was assessed through evaluations of adverse events (AEs) and local tolerability [erythema, scaling, dryness, and stinging/burning on scales ranging from 0 (none) to 3 (severe)].

A total of 142 subjects were randomized to adapalene-BPO and 143 to vehicle. At study endpoint (week 12), adapalene-BPO was significantly superior to vehicle in terms of treatment success (about 49.3% vs. about 15.9%, respectively), and regarding percentage reduction in total lesion counts (about 68.6% vs. about 19.3%), inflammatory (about 63.2% vs. about 14.3%) and non-inflammatory lesion counts (about 70.7% vs. about 14.6%) (all p<0.001). More subjects using adapalene-BPO reported that their acne had no effect on their quality of life, and parents noted that their child's acne significantly improved. Adapalene-BPO was well-tolerated, with mean tolerability scores less than 1 (mild).

In preadolescents with acne, adapalene-BPO leads to significantly superior treatment success and lesion count reduction compared to vehicle.

### INTRODUCTION

The epidemiology of acne vulgaris is evolving, with an increasingly earlier onset seen in patients 8-11 years old. Analysis of U.S. national data over approximately the last thirty years indicates a significant decrease in the age of children presenting for acne visits, with the greatest increase in the early acne group. There is growing consensus about age groupings in pediatric acne. Preadolescent acne without underlying endocrinopathy is now considered by clinicians as a normal variant, comprised of children 7-11 years old. Acne affects all age groups, although peak incidence is at puberty due to increased androgen production. Acne in adolescents may persist another 8-12 years, with about 7% continuing into the third and fourth decade. Therefore, controlling acne early on may help to minimize impact over the lifespan of disease. Furthermore, an early onset of acne is associated with worsened disease severity in later years, as demonstrated by a 5-year cohort study of young girls who had early development of significant comedonal acne. In a prospective study of children aged 5-12 years, sebum production and Propionibacterium acnes(P. acnes) increased earlier in both prepubertal and pubertal children who developed acne compared with children who did not. Early onset of acne is also a risk factor for scarring, with delayed treatment leading to worse scars.

Treatment is always indicated when the clinician suspects current or potential scarring or psychological morbidity, even in younger patients with mild acne and few comedones.

Acne treatments need to be studied in the preadolescent population, as most acne medications are indicated for patients 12 years of age and older.

In the current study, our objective was to evaluate the efficacy and safety of adapalene-BPO administered for up to about 12 weeks in subjects about 9 to about 11 years old with acne vulgaris.

### I—Patients and Methods

### A) Study Design

This was a multicenter, randomized, vehicle-controlled, double-blind study conducted at 20 centers in the U.S. and 5 centers in Canada between Jun. 21, 2010, and Aug. 2, 2011. Subjects were randomized in a 1:1 ratio to receive adapalene-BPO fixed-dose combination gel (Epiduo®Gel, Galderma Laboratories) or vehicle gel once daily in the evening to the face and trunk (if applicable) for up to 12 weeks. Integrity of the blinding was ensured by packaging the medication in identical tubes and requiring a third party other than the investigator to dispense the medications. Only the designated personnel directly responsible for labelling the study medications had access to the randomization lists. Subjects were also instructed to use a moisturizer throughout the study (Cetaphil® Moisturizing Lotion or subject's moisturizer). Study visits were performed at weeks 1, 2, 4, 8 and 12. The study was conducted in accordance with the ethical principles derived from the Declaration of Helsinki revised version (Somerset West, 1996), the International Conference on Harmonization (ICH) Good Clinical Practice (GCP), under provisions of the Pediatric Research Equity Act, and in compliance with local regulatory requirements. The study was reviewed and approved by institutional review boards/ethics committees. Prior to enrollment, the subject and their parent/legal representative provided their written informed consent.

### B) Patient Selection

Enrolled subjects were male or female, 9 to 11 years of age, with a score of 3 (moderate) on the Investigator's Global Assessment (IGA) scale and 20-100 total lesions (non-inflammatory and/or inflammatory) on the face, including the nose. Subjects with truncal acne vulgaris could also participate. Specified washout periods were required for subjects taking certain topical and systemic treatments. Subjects were excluded if they had acne nodules or cysts, severe acne requiring systemic treatment, or if they used hormonal contraceptives.

### C) Efficacy Assessments

Efficacy assessments were performed on facial lesions. The primary outcome measures were success rate, defined as the percentage of subjects rated “clear” or “almost clear” with at least 2 grades reduction from baseline on the IGA at each visit, and the change from baseline in total lesion counts at week 12.

As detailed in Table 1 hereunder, it is important to note that an IGA scale modified for pediatrics was used in this study, rated on a scale of 0 (clear—no comedones, papules or pustules) to 4 (severe—widespread and numerous comedones with many small, medium sized and large papules and pustules).

TABLE 1
Comparison between Modified and Standard IGA Scales

| | | MODIFIED | STANDARD |
|---|---|---|---|
| 0 | Clear | No comedones, papules or | Residual hyperpigmentation and |
| | | pustules | erythema may be present. |
| | | Residual hyperpigmentation | |
| | | and erythema may be present. | |
| 1 | Almost | Rare comedones | A few scattered comedones and a few |
| | Clear | No more than a few small | small papules |
| | | papules and pustules | |
| 2 | Mild | Easily recognizable comedones | Easily recognizable |
| | | in limited numbers | Less than half the face is involved |
| | | ±Presence of some small | Some comedones, papules and |
| | | papules or pustules | pustules |
| 3 | Moderate | Many comedones | More than half of the face is |
| | | ±Easily recognizable small and | involved |
| | | medium-sized papules | Many comedones, papules and |
| | | No nodules or cysts. | pustules |
| | | | One nodule may be present |
| 4 | Severe | Widespread and numerous | Entire face is involved |
| | | comedones | Covered with comedones, |
| | | Many small, medium-sized and | numerous papules & pustules |
| | | large papules and pustules | Few nodules and cysts |
| | | Nodules or cysts may or may | |
| | | not be present. | |

Secondary outcome measures were the median percentage changes from baseline in total, inflammatory and non-inflammatory lesion counts at each visit. Also, the Children's Dermatology Life Quality Index (C-DLQI) was evaluated at baseline and week 12. This is a dermatology-specific Quality of Life instrument, ranging from 0-30, with higher scores representing a more impaired quality of life. Lastly, the Parent/Legal Representative Assessment of Acne (hereafter called Parent Assessment of Acne) was performed at week 12, evaluating the child's facial acne on a scale of 0 (complete improvement) to 5 (worse).

### D) Safety Assessments

Safety was assessed through evaluations of adverse events (AEs) and local tolerability [including erythema, scaling, dryness, and stinging/burning on scales ranging from 0 (none) to 3 (severe)].

### E) Statistical Analyses

Efficacy was evaluated on the intent-to-treat (ITT) population, including randomized subjects who received the study drug, using the last observation carried forward (LOCF) method to impute missing values. For the purpose of this publication, reference is also made to observed data. Efficacy analyses were repeated on the per protocol (PP) population to confirm these results. Safety was evaluated on the safety population, including randomized subjects who applied the study drug at least once. The success rate was analyzed using the Cochran Mantel Haenszel (CMH) test stratified by analysis center, using general association. The median percent changes in lesion counts were analyzed by the CMH test row mean difference statistic using Relative to the Identified Distribution (RIDIT) score, stratified by analysis center. Analysis of the Parent Assessment of Acne was performed using the CMH test row mean difference statistic using RIDIT score stratified by analysis center, and the C-DLQI data were summarized by descriptive statistics.

All tests were conducted two-sided, each at the 0.05 significance level. The sample size calculation was based on previous studies, assuming a 17.3% difference between groups in success rate. For this, 284 randomized subjects were required, based on a 0.05 significance level and 90% power, with a drop-out rate of 15%.

### II—Results

### A) Subject Disposition and Demographics

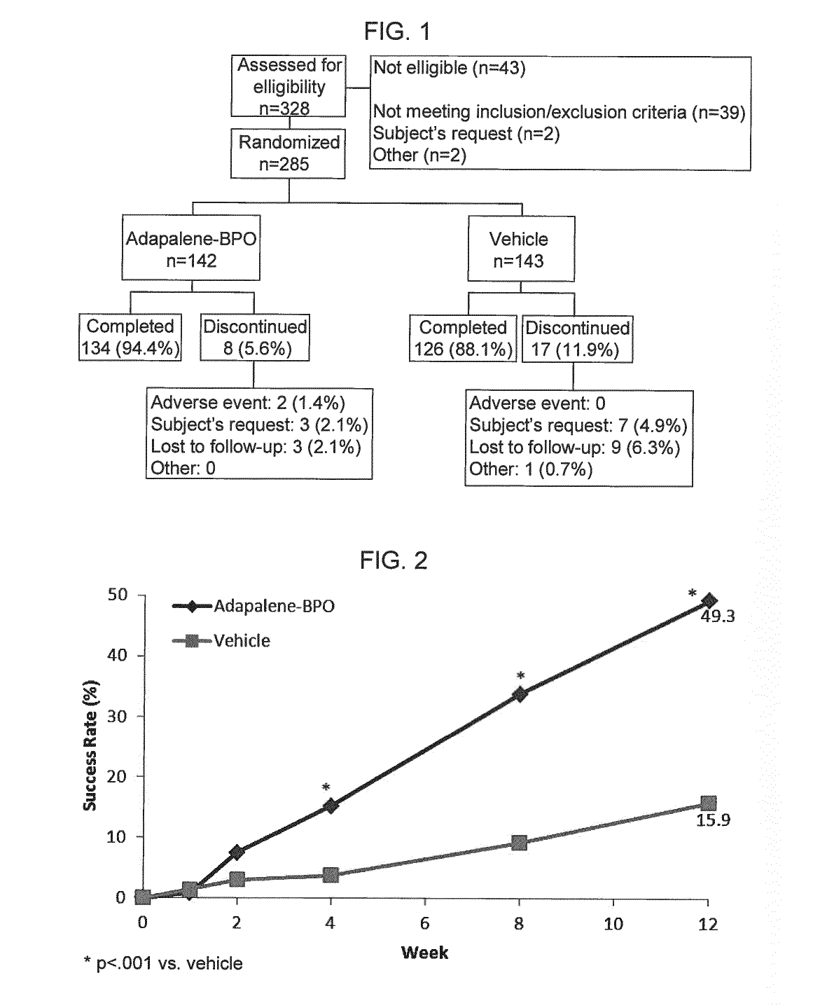

A total of 285 subjects were randomized in this study: 142 to adapalene-BPO and 143 to vehicle (FIG. 1). More subjects had normal completion in the adapalene-BPO group (94.4% vs. 88.1% for vehicle). Demographic and baseline disease characteristics were similar between groups (Table 2), although there was a higher total lesion count for vehicle than adapalene-BPO (56.4 vs. 50.5, respectively, p=0.015; considered in statistical analysis). The majority of subjects were female (76.1%) and Caucasian (58.9%), and all were deemed to have moderate acne. The mean inflammatory lesion count was 15.2 and non-inflammatory count was 38.3 (12 and 34 for median counts, respectively), consistent with the typical presentation of acne in younger individuals characterized by a predominance of non-inflammatory lesions. Subjects had acne for an average duration of 1.72 years.

TABLE 2
Demography and Baseline Characteristics

| | Adapalene- | | |
|---|---|---|---|
| | BPO | Vehicle | Total |
| | (N = 142) | (N = 143) | (N = 285) |
| Gender, n (%) | | | |
| Male | 33 (23.2) | 35 (24.5) | 68 (23.9) |
| Female | 109 (76.8) | 108 (75.5) | 217 (76.1) |
| Age, Year | | | |
| Mean ± SD | 10.3 ± 0.76 | 10.4 ± 0.68 | 10.4 ± 0.72 |
| 9 years old, % | 17.6 | 10.5 | 14.0 |
| 10 years old, % | 31.7 | 34.3 | 33.0 |
| 11 years old, % | 50.7 | 55.2 | 53.0 |
| Race, n (%) | | | |
| Caucasian | 81 (57.0) | 87 (60.8) | 168 (58.9) |
| Black | 36 (25.4) | 32 (22.4) | 68 (23.9) |
| Asian | 2 (1.4) | 1 (0.7) | 3 (1.1) |
| Hispanic | 6 (4.2) | 5 (3.5) | 11 (3.9) |
| Other | 17 (12.0) | 18 (12.6) | 35 (12.3) |
| Investigator's Global | | | |
| Assessment (IGA), n (%) | | | |
| 3 = Moderate | 142 (100) | 143 (100) | 285 (100) |
| Mean Lesion Counts | | | |
| Total | 50.5 | 56.4 | 53.5 |
| Inflammatory | 13.8 | 16.6 | 15.2 |
| Non-inflammatory | 36.7 | 39.9 | 38.3 |
| Truncal lesions, n (%) | 28 (19.7) | 34 (23.8) | 62 (21.8) |

### B) Efficacy

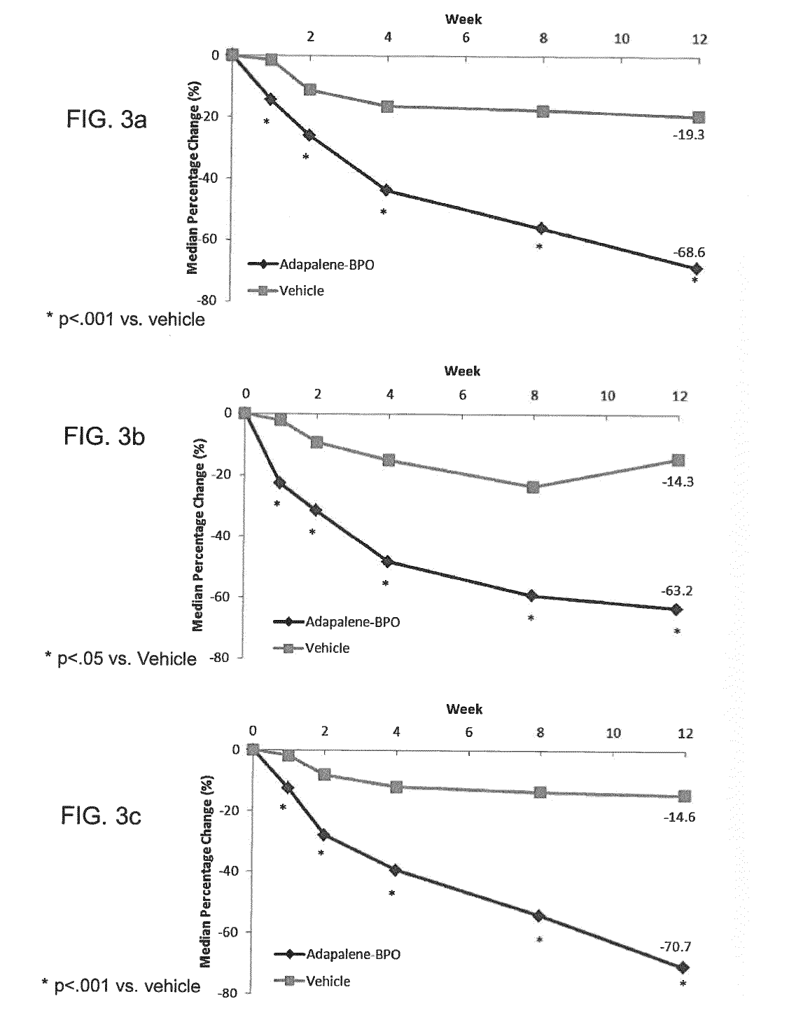

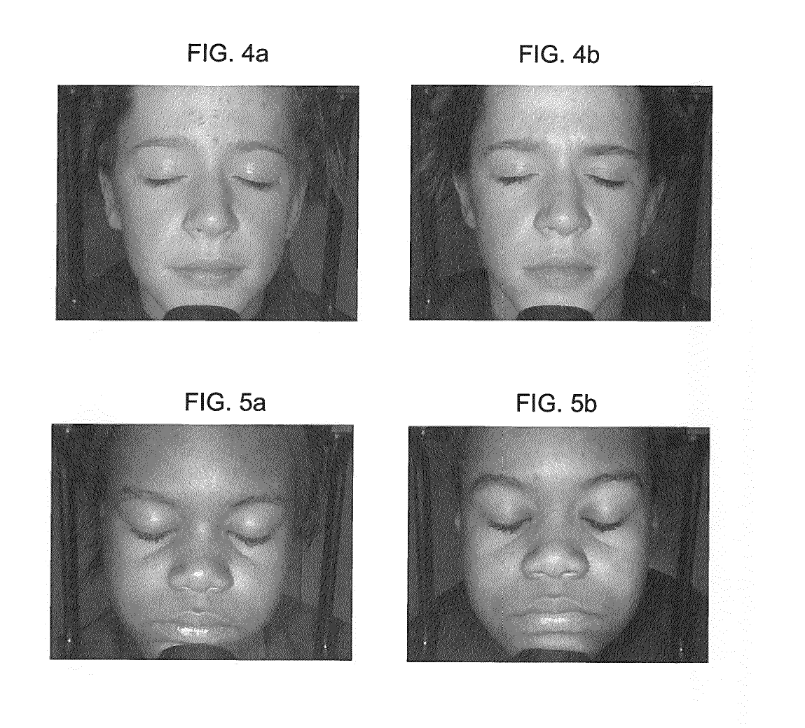

At study endpoint (week 12), adapalene-BPO was significantly superior to vehicle in terms of treatment success: nearly half of the subjects (about 49.3%) vs. about 15.9% of subjects, respectively, were rated “clear” or “almost clear” (p<0.001; FIG. 2), confirmed by PP analyses. For adapalene-BPO, the success rate increased continuously throughout the study, reaching significance as early as week 4 (p<0.001). Adapalene-BPO was significantly more effective than vehicle regarding the change from baseline in total lesion counts as early as week 1 (p<0.001). At week 12, adapalene-BPO was also significantly more effective than vehicle in terms of median percentage reduction (about 68.6% vs. about 19.3%, p<0.001; FIG. 3A). This was also true for percentage reduction of both inflammatory (about 63.2% vs. about 14.3%) and non-inflammatory lesion counts (about 70.7% vs. about 14.6%) at the end of the study (both p<0.001; FIGS. 3B and C). Regardless of the lesion type, adapalene-BPO showed a significant decrease in lesion counts as early as week 1 (p<0.05). For the vehicle, however, percentage change in total and non-inflammatory lesions plateaued, and worsened (increased) for inflammatory lesions between weeks 8 and 12. Photographs of subjects in FIGS. 4 and 5 depict the improvement seen after 12 weeks of adapalene-BPO use.

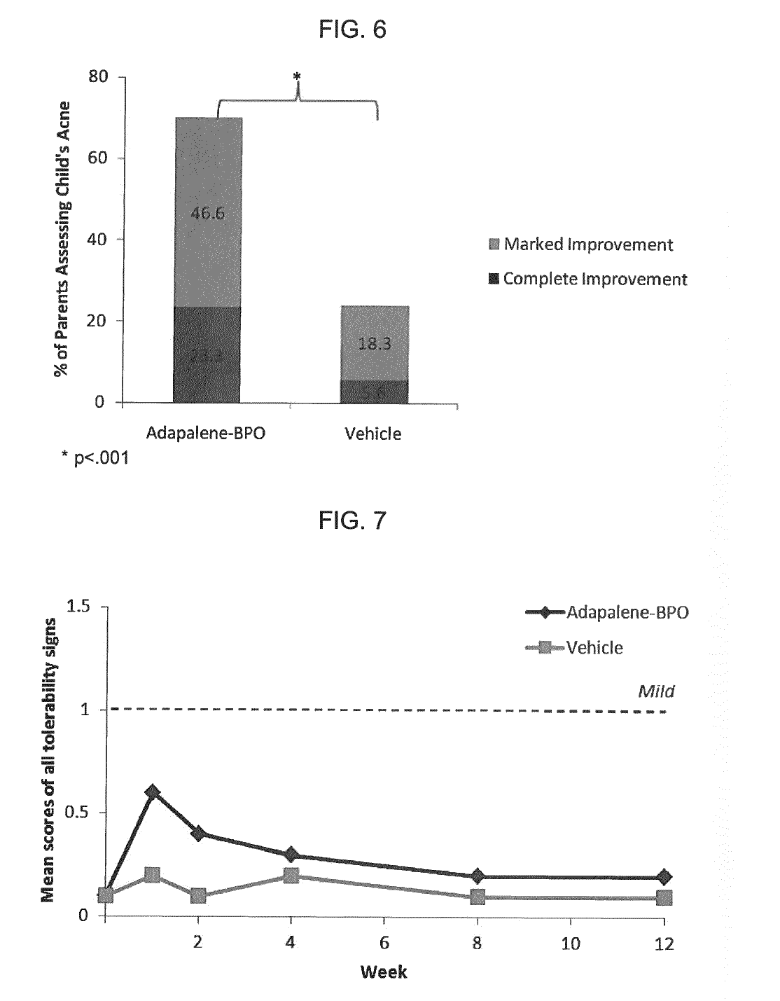

Regarding the C-DLQI scores at week 12, more subjects in the adapalene-BPO group compared to vehicle reported that their acne had no effect on their quality of life (about 71.0% vs. about 57.5%, respectively). Also, significant differences were found between groups in favor of adapalene-BPO for the Parent Assessment of Acne at week 12, with about 69.9% of subjects in the adapalene-BPO group showing complete or marked improvement vs. about 23.9% in the vehicle group (p<0.001; FIG. 6).

### C) Safety

Adapalene-BPO was generally well-tolerated (FIG. 7). Mean scores and mean worst scores for erythema, scaling, dryness and stinging/burning did not exceed 1 (mild) for both groups throughout the study. Though mean scores peaked for adapalene-BPO in the first two weeks of treatment, this remained mild and disappeared over time to reach a tolerability level comparable to vehicle as of week 4. There were 29 subjects (20.4%) who experienced treatment-related AEs in the adapalene-BPO group, and 1 subject (0.7%) in the vehicle group. All related AEs were dermatologic in nature, the most common being skin burning sensation (9.2%) and skin irritation (5.6%). Two AEs (1.4% of subjects) in the adapalene-BPO group led to discontinuation (erythema and skin irritation, both mild in severity). It is of note that most AEs were transient and mild or moderate in severity, with 1 subject in each group who had a severe AE (1 related skin irritation in the adapalene-BPO group, which resolved without treatment).

### D) Discussion

This is one of few studies to explore the treatment of pediatric acne, particularly in the preadolescent age group of about 9-11 years old. As the onset of acne tends to be earlier, pediatricians are likely to see this as an increasingly common presentation in younger age groups that may worsen with time if left untreated. Indeed, while preadolescents frequently develop follicular plugs and comedones, advances in acne pathophysiology demonstrate that P. acnes proliferation and inflammation generally will develop later. Scarring is typically considered to be the consequence of inflammatory lesions, but even comedonal acne may be related to scarring.

Prompt treatment of acne with a fast onset of action may help to not only prevent scarring, but also later severity progression in older childhood and adolescent years, and possibly future decades of acne cycles into adulthood. In our study among pediatric patients with moderate acne, adapalene-BPO achieved a significantly higher treatment success compared to vehicle as early as week 4 (p<0.001). Adapalene-BPO was also significantly more effective in reducing both inflammatory and non-inflammatory lesions at week 12, with a median percent reduction of about 63.2% and about 70.7% (p<0.001 vs. vehicle). In addition, for total, inflammatory and non-inflammatory lesions, results were significantly superior for adapalene-BPO as early as week 1, and remained significant at all time points (FIG. 3).

Previous studies of topical retinoids in pre-adolescents are limited. BPO has a long history of use in acne, both as a single agent for acne or paired with a topical retinoid for the treatment of mild to moderate acne. This study displays the use of retinoid-BPO combination therapy, targeting multiple pathways of acne pathogenesis with a single topical agent, utilizing an antimicrobial agent not associated with development of bacterial resistance.

The paediatric population included in this study was characterized by having a predominance of non-inflammatory lesions, as consistent with the literature. Following discussions with paediatric dermatologists, the IGA scale utilized was adapted to the specific clinical presentation of preadolescents, and differs from IGA scales which were utilized for other studies of adapalene-BPO and other topical acne medications. Our results are strengthened by utilizing other measures of acne impact, showing that more subjects using adapalene-BPO reported that their acne had no effect on their quality of life as compared to those on vehicle. Also, parents/caregivers noted that their child's acne had a significantly higher complete or marked improvement after adapalene-BPO treatment compared to vehicle. The objective treatment success and high ratings from parents in our study may partially reflect the ease of adherence to the once daily use regimen of adapalene-BPO. Since even medically mild acne may be perceived by pediatric patients as extremely distressing, a careful assessment of a child's emotional health as well as potential effects on the family is appropriate, and should influence acne management.

### CONCLUSION

The treatment of preadolescent acne remains an area of paediatrics to be further explored. Results of this randomized controlled trial add evidence that early treatment of acne is reasonable and well-tolerated in this population, and can minimize acne complications including disease progression and scarring. Twelve weeks of adapalene-BPO treatment led to significantly superior treatment success compared to vehicle, superior reduction from baseline in lesion counts, good parent evaluation and trends toward an improved quality of life.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A method of treating preadolescent acne, the method comprising administering an effective amount of a pharmaceutical composition comprising a fixed combination of adapalene and benzol peroxide to a preadolescent patient in need thereof, wherein the preadolescent patient is from age 7 to age 11, and wherein the pharmaceutical composition comprises from 0.01% to 2% by weight of adapalene and from 0.1% to 5% by weight by weight of benzol peroxide.

2. The method of claim 1, wherein the pharmaceutical composition comprises from 0.05% to 0.5% by weight of adapalene.

3. The method of claim 1, wherein the pharmaceutical composition comprises from 0.5% to 5% by weight of benzol peroxide.

4. The method of claim 1, wherein the pharmaceutical composition comprises from 0.1% to 0.3% by weight of adapalene and 2.5% by weight of benzol peroxide.

5. The method of claim 1, wherein the pharmaceutical composition is formulated as a topical gel.

6. The method of claim 5, wherein the topical gel consists of the following formulation in which percents of each ingredient are expressed by weight with regard to the total weight of the composition:

| | |
|---|---|
| Adapalene | 0.10%; |
| Benzoyl peroxide | 2.50%; |
| Copolymer of acrylamide and sodium | 4.00%; |
| acryloyldimethyltaurate in isohexadecane and | |
| polysorbate 80 | |
| Sodium docusate | 0.05%; |
| Glycerol | 4.00%; |
| Poloxamer | 0.20%; |
| Propylene glycol | 4.00%; and |
| Purified water | qs 100%. |

7. The method of claim 1, wherein the pharmaceutical composition is topically administered to the preadolescent patient for a period of 12 weeks.

8. The method of claim 1, wherein the method results in a treatment success of at least 49%.

9. The method of claim 1, wherein the method results in a reduction in total lesion count of at least 68%.

10. The method of claim 1, wherein the method results in a reduction of inflammatory lesion count of at least 63%.

11. The method of claim 1, wherein the method results in a reduction of non-inflammatory lesion count of at least 70%.

12. The method of claim 1, wherein the method provides improved treatment success when compared with vehicle alone as early as week 4 of treatment.

13. The method of claim 1, method results in total inflammatory and non-inflammatory lesions results were significantly superior as early as week 1 when compared with vehicle alone.

14. A method of treating non-inflammatory acne lesions in a preadolescent patient, the method comprising topically administering to the skin of the preadolescent patient an effective amount of a fixed combination comprising from about 0.1% to about 0.3% by weight adapalene and about 2.5% by weight BPO, wherein the preadolescent patient is from about age 7 to about age 11 and wherein the amount of the fixed combination administered is effective to result in a reduction of non-inflammatory lesion count of at least about 70%.

15. The method of claim 14, wherein the fixed combination is formulated as a topical gel.

16. The method of claim 15, wherein the topical gel consists of the following formulation in which percents of each ingredient are expressed by weight with regard to the total weight of the composition:

| | |
|---|---|
| Adapalene | 0.10%; |
| Benzoyl peroxide | 2.50%; |
| Copolymer of acrylamide and sodium | 4.00%; |
| acryloyldimethyltaurate in isohexadecane and | |
| polysorbate 80 | |
| Sodium docusate | 0.05%; |
| Glycerol | 4.00%; |
| Poloxamer | 0.20%; |
| Propylene glycol | 4.00%; and |
| Purified water | qs 100%. |

17. The method of claim 14, wherein the fixed combination is topically administered to the preadolescent patient for a period of about 12 weeks.

18. The method of claim 14, wherein the method results in a treatment success of at least about 49%.

19. The method of claim 14, wherein the method results in a reduction in total lesion count of at least about 68%.

20. The method of claim 14, wherein the method results in a reduction of inflammatory lesion count of at least about 63%.

21. The method of claim 14, wherein the method provides improved treatment success when compared with vehicle alone as early as week 4 of treatment.

22. A pharmaceutical composition comprising a fixed combination of adapalene and benzol peroxide, for its use in a method of treating preadolescent acne which comprises topically administering said pharmaceutical composition to a preadolescent patient in need thereof, wherein the preadolescent patient is from age 7 to age 11, and wherein the pharmaceutical composition comprises from 0.01% to 2% by weight of adapalene and from 0.1% to 5% by weight by weight of benzol peroxide.

23. The pharmaceutical composition of claim 22, wherein said composition comprises 0.05% to 0.5% by weight of adapalene.

24. (canceled)

25. The method of claim 2, wherein the pharmaceutical composition comprises from 0.1% to 0.3% by weight of adapalene.

26. The method of claim 2, wherein the pharmaceutical composition comprises from 0.1% by weight of adapalene.

27. The method of claim 3, wherein the pharmaceutical composition comprises from 2% to 5% by weight of benzol peroxide.

28. The method of claim 3, wherein the pharmaceutical composition comprises from 2.5% by weight of benzol peroxide.

29. The method of claim 4, wherein the pharmaceutical composition comprises from 0.1% by weight of adapalene and 2.5% by weight of benzol peroxide.

30. The pharmaceutical composition of claim 22, wherein the pharmaceutical composition comprises from 0.5% to 5% by weight of benzol peroxide.

31. The pharmaceutical composition of claim 22, wherein the pharmaceutical composition comprises from 0.1% to 0.3% by weight of adapalene and 2.5% by weight of benzol peroxide.

32. The pharmaceutical composition of claim 22, wherein the pharmaceutical composition is formulated as a topical gel.

33. The pharmaceutical composition of claim 22, wherein the topical gel consists of the following formulation in which percents of each ingredient are expressed by weight with regard to the total weight of the composition:

| | |
|---|---|
| Adapalene | 0.10%; |
| Benzoyl peroxide | 2.50%; |
| Copolymer of acrylamide and sodium | 4.00%; |
| acryloyldimethyltaurate in isohexadecane and | |
| polysorbate 80 | |
| Sodium docusate | 0.05%; |
| Glycerol | 4.00%; |
| Poloxamer | 0.20%; |
| Propylene glycol | 4.00%; and |
| Purified water | qs 100%. |
